(19) United States
(12) Patent Application Publication
(10) Pub. No.: US 2003/0103993 A1
(43) Pub. Date: Jun. 5, 2003

(54) HLA-DR2 binding peptides

(75) Inventors: Shrikant Deshpande, Fremont, CA (US); Subhashini Arimilli, Fremont, CA (US); Kalidip Choudhury, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation, Seattle, WA (US)

(21) Appl. No.: 10/012,363

(22) Filed: Dec. 12, 2001

Related U.S. Application Data
(60) Provisional application No. 60/254,886, filed on Dec. 12, 2000.

(51) Int. Cl. A61K 38/17; A61K 39/00; C07K 14/435; C07K 7/08; A61K 9/127

(52) U.S. Cl. 424/185.1; 424/450; 530/324

(57) ABSTRACT

The invention provides a polypeptide sequence, GHIKSSISFMGM, that disrupts binding of the amino acid sequence peptides of myelin basic protein (MBP) to HLA-DR2 class II MHC molecules and acts as an antagonist in DR2-restricted antigen presentation to human T cell clones. In particular, the invention provides the polypeptide sequence itself and variants, compositions comprising the polypeptide, polynucleotides encoding the polypeptide, and methods for using the polypeptides and polynucleotides for treating autoimmune disorders.

### CROSS-REFERENCES TO RELATED APPLICATIONS

The present application claims priority to U.S. S No. 60/254,886, filed Dec. 12, 2000, herein incorporated by reference in its entirety.

### STATEMENT AS TO RIGHTS TO INVENTIONS MADE UNDER FEDERALLY SPONSORED RESEARCH AND DEVELOPMENT

Not applicable.

### FIELD OF INVENTION

The present invention relates generally to therapy of autoimmune disorders, such as multiple sclerosis. The invention is more specifically related to a peptide, comprising GHIKSSISFMGM (SEQ ID NO:1), which competitively inhibits binding of the amino acid sequence peptides of myelin basic protein (MBP) to class II MHC HLA-DR2 molecules. Such a peptide is useful for the treatment of autoimmune disorders.

### BACKGROUND OF THE INVENTION

More than 30 autoimmune diseases are presently known; these include many which have received much public attention, including myasthenia gravis (MG) and multiple sclerosis (MS). Characteristic of these disease is the attack by the immune system on the tissues of the host. A crucial element of the immune system is the MHC molecule, which is expressed on accessory cells and presents short peptide fragments of both self and foreign proteins to T cells. In non-diseased individuals the immune system recognizes self antigens and does not mount an immune response. However, in diseased individuals self peptides are not recognized and are treated like foreign antigen by autoreactive T cells.

Antigens for a number of autoimmune diseases are known. For example, in experimentally induced autoimmune diseases, antigens involved in pathogenesis have been characterized: native type II collagen is identified in collagen-induced arthritis and mycobacterial heat shock protein in adjuvant arthritis in rat and mouse (Stuart et al., Ann. Rev. Immunol. 2:199-218 (1984); van Eden et al., Nature 331:171-173 (1988)); thyroglobulin has been identified in experimental allergic thyroiditis (EAT) in mice (Marion et al., J. Exp. Med. 152:1115-1120 (1988)); acetylcholine receptor (AChR) in experimental allergic myasthenia gravis (EAMG) (Lindstrom et al., Adv. Immunol. 42:233-284 (1988)); and myelin basic protein (MBP) and proteolipid protein (PLP) in experimental allergic encephalomyelitis (EAE) in mouse and rat (Acha-Orbea et al., Ann. Rev. Imm. 7:377-405 (1989)). In addition, target antigens have been identified in humans: type II collagen in human rheumatoid arthritis (Holoshitz et al., Lancet ii:305-309 (1986)), acetylcholine receptor in myasthenia gravis (Lindstrom et al., Adv. Immunol. 42.:233-284 (1988)) and myelin basic protein in multiple sclerosis (Acha-Orbea et al., Ann. Rev. Imm. 7:377-405 (1989)).

Specific autoimmune diseases are correlated with certain MHC types. For example, HLA-DR2⁺ and HLA-DR3⁺ individuals are at a higher risk than the general population to develop systemic lupus erythematosus (SLE) (Reinertsen et al., N. Engl. J. Med. 299:515-18 (1970)). Myasthenia gravis has been linked to HLA-D (Safwenberg et al., Tissue Antigens 12:136-42 (1978)). Susceptibility to rheumatoid arthritis is associated with HLA-D/DR in humans. Exemplary alleles for IDDM include DR4, DQ8, DR3, DQ3.2. Methods for identifying which alleles, and subsequently which MHC-encoded polypeptides, are associated with an autoimmune disease are known in the art.

Current treatments for autoimmune disease and related conditions consist primarily of treating the symptoms, but not intervening in the etiology of the disease. Broad spectrum chemotherapeutic agents are typically employed and are often associated with numerous undesirable side effects such as nonspecific suppression of an individual's overall immune response. Therefore, there is a need for compounds capable of selectively suppressing autoimmune responses by blocking MHC binding, thereby providing a safer, more effective treatment. A desirable approach would be to use a peptide which competitively blocks binding to class II MHC molecules. The present invention fulfills such needs, and provides related advantages.

### SUMMARY OF THE INVENTION

Within a first aspect the present invention provides an isolated polypeptide comprising an amino acid sequence of SEQ ID NO:1.

Within one embodiment, the polypeptide is from 25 to 50 amino acids in length.

Within another embodiment, the polypeptide is from 50 to 100 amino acids in length.

Within yet another embodiment, the polypeptide has an amino acid sequence of SEQ ID NO:1

Within yet another embodiment, the polypeptide comprises post-translational modifications.

Within a second aspect, the invention provides a pharmaceutical composition comprising a pharmaceutically acceptable carrier and a polypeptide comprising an amino acid sequence of SEQ ID NO:1.

Within one embodiment, the composition further comprises a member selected from the group consisting of: a) liposome, b) nanocapsule, and c) microparticle.

Within a third aspect, the invention comprises a method of treating autoimmune disease in a subject, the method comprising the step of administering to the subject a therapeutically effective amount of a polypeptide comprising an amino acid sequence of SEQ ID NO:1.

Within one embodiment, the subject is a human.

Within another embodiment, the autoimmune disease is associated with HLA-DR class II MHC molecules.

Within another embodiment, the HLA-DR class II MHC molecules are HLA-DR2 molecules.

Within yet another embodiment, the autoimmune disease is a member selected from the group consisting of: a) insulin-dependent diabetes mellitus, b) multiple sclerosis, c) mysathenia gravis, d) pernicious anemia, e) rheumatoid arthritis, and f) systemic lupus erythematosus.

Within another embodiment, the polypeptide is administered with a pharmaceutically acceptable carrier.

Within a further embodiment, the polypeptide comprises an epitope that competes with the myelin basic protein (MBP) protein for binding.

Within still another embodiment, the polypeptide comprises an epitope that antagonizes the T cell response induced by myelin basic protein (MBP) protein.

In a fourth aspect, the invention further provides an isolated polypeptide comprising an amino acid sequence having at least 90% identity to the sequence provided in SEQ ID NO:1.

In a fifth aspect, the invention further provides a polynucleotide encoding a polypeptide comprising the amino acid sequence of SEQ ID NO:1.

Within one embodiment, a method of treating autoimmune disease in a subject comprises the step of administering to the subject a therapeutically effective amount of the polynucleotide.

In another embodiment, the invention further provides a polypeptide comprising the amino acid seuqence of SEQ ID NO:1 bound to the antigen binding pocket of an MHC class II molecule comprising an β1 domain and a α1 domain. In another embodiment, the MHC class II molecule comprises an a β1-β2 domain α1-α2 domain. In another embodiment, the MHC class II molecule comprises a β chain and an α chain. In one embodiment, the polypeptide and the MHC class II are covalently bound. In one embodiment, the polypeptide and the MHC class II are non-covalently bound. In one embodiment, the MHC class II molecule is recombinant. In another embodiment, the MHC class II molecule is a single chain molecule, comprising an β1 domain and a α1 domain linked by an amino acid linker.

These and other aspects of the invention will become evident upon reference to the following detailed description.

### BRIEF DESCRIPTION OF THE DRAWINGS

FIG. 1. Competitive binding of the MBP peptide versus the test peptides.

FIG. 2. Acidification rates of SS8T cells in response to MBP in the presence of test peptide.

FIG. 3. γ-Interferon response of SS8T cells in response to MBP in the presence of test peptide.

### DETAILED DESCRIPTION OF THE INVENTION

A. Introduction

The present invention provides a polypeptide sequence, GHIKSSISFMGM, and methods of identifying polypeptide sequences that disrupt binding of specific epitopes with class II MHC molecules. As class II MHC molecules play a critical role in the immune response, this invention has applications in any situation where it is desirable to depress certain components of the immune defense system.

Since inappropriate immune response is a central mechanism of pathogenesis and progression of autoimmune diseases, this invention is particularly directed towards treatment of such diseases. In autoimmune disorders, autoantigenic peptides bind to disease-associated class II MHC molecules and are presented to autoreactive T cells. Several autoimmune disorders are known to be associated with HLA-DR alleles of class II MHC molecules. Compounds, including peptides, that bind to disease-associated class II MHC and prevent this antigen presentation are expected to have therapeutic effects on the disease.

Therefore, in preferred embodiments, the peptide GHIKSSISFMGM disrupts binding of autoantigens with HLA-DR class II MHC molecules and is used in methods to treat autoimmune disorders. Further, in certain embodiments, the autoantigen is myelin basic protein (MBP) and the HLA-DR class II MHC molecule is the DR2 allele. MBP is an autoantigen in experimentally induced encephalitis (EAE), a model for multiple sclerosis. In humans, multiple sclerosis has been shown to be associated with HLA-DR2 molecules. As such, the present invention will be particularly useful for the treatment of multiple sclerosis and other HLA-DR2-associated autoimmune disorders.

Finally, the invention also provides variants of the polypeptide sequence, pharmaceutical compositions comprising the polypeptide, polynucleotides encoding the polypeptide, and methods for using polynucleotides encoding the polypeptide for treating autoimmune disorders.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook et al., Molecular Cloning: A Laboratory Manual (2nd Ed., 1989); Maniatis et al., Molecular Cloning: A Laboratory Manual (1982); DNA Cloning: A Practical Approach vol. I & II (Glover, ed.); Oligonucleotide Synthesis (Gait, ed., 1984); Nucleic Acid Hybridization, (Hames & Higgins, eds., 1985); Transcription and Translation (Hames & Higgins, eds., 1984); Animal Cell Culture (Freshney, ed., 1986); Perbal, Practical Guide to Molecular Cloning (1984).

B. Definitions

Prior to setting forth the invention, it may be helpful to an understanding thereof to provide definitions of certain terms to be used hereinafter:

An “antigen” is a molecule or fragments thereof that provokes an immune response.

An “autoantigen” is an antigen or fragment thereof derived from a “self” molecule which contains an epitope recognized by immune cells.

An “epitope” is a site on an antigen that is recognized and bound by a particular antibody, B cell receptor, T cell receptor, class I MHC molecule, or class II MHC molecule.

“Myelin basic protein (MBP)” is the autoantigen for experimental allergic encephalomyelitis, an autoimmune disorder in animals that is the model for the human disease multiple sclerosis. It is also an autoantigen for multiple sclerosis.

“HLA-DR molecules” are one of the three major groups of class II MHC proteins encoded for by different genetic loci. For example, an HLA-DR2 molecule is one particular allele of the HLA-DR class II MHC molecules.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “DNA” and “polynucleotide” are used essentially interchangeably herein to refer to a DNA molecule that has been isolated free of total genomic DNA of a particular species. “Isolated,” as used herein to refer to DNA molecules means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

“Polynucleotides” of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses “conservatively modified variants” thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Conservatively modified variants” also refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “polypeptide” is used in its conventional meaning, i.e,. as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-translational modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

A polypeptide may also be a fusion polypeptide that comprises multiple polypeptides as described herein. A fusion partner may, for example, assist in expressing the protein (an expression enhancer) at higher yields than the native recombinant protein. Other fusion partners may be selected so as to increase the solubility of the polypeptide or to enable the polypeptide to be targeted to desired intracellular compartments. Still further fusion partners include affinity tags, which facilitate purification of the polypeptide.

An “isolated” polypeptide is one that is removed from its original environment. For example, a naturally-occurring protein or polypeptide is isolated if it is separated from some or all of the coexisting materials in the natural systems.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Unless otherwise indicated, a particular amino acid sequence also implicitly encompasses “conservatively modified variants” thereof. One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides, when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see, Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptide of SEQ ID NO:1 can be made detectable, e.g., by incorporating a radiolabel into the peptide)

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a nucleic acid. As used herein, a promoter includes necessary nucleic acid sequences near the start site of transcription, such as, in the case of a polymerase H type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

As used in this specification and the appended claims, the singular forms “a,” “an” and “the” include plural references unless the content clearly dictates otherwise.

C. MHC Molecules

Class II MHC molecules are one of two classes of highly polymorphic proteins encoded by the major histocompatibility complex (MHC), both of which are membrane-associated and present antigen to T lymphocytes (T cells). Class I and class II MHC molecules are distinguished by the types of cells on which they are expressed, and by the type of immune response evoked by the presented antigen. Class I MHC molecules (e.g., HLA-A, -B and -C molecules in the human system) are expressed on almost all nucleated cells and are recognized by cytotoxic T lymphocytes (CTL), which then destroy the antigen-bearing cells. In constrast, class II MHC molecules (HLA-DP, -DQ and -DR, for example, in humans) are expressed primarily on the surface of antigen-presenting cells, such as B lymphocytes, dendritic cells, macrophages and recognized by CD4⁺ T helper lymphocytes (T_{H}). T_{H} cells induce proliferation of both B and T lymphocytes, thus amplifying the immune response to the particular antigenic peptide that is displayed (Takahashi, Microbiol. Immunol. 37:1-9, (1993)).

Antigens presented by these two molecules are obtained from two distinct processing pathways. Intracellular antigens, synthesized inside of the cell, such as from viral or newly synthesized cellular proteins, for example, are processed and presented by class I MHC. Exogenous antigens, taken up by the antigen-presenting cell (APC) from outside of the cell through endocytosis, are processed and presented by class II MHC. After the antigenic material is proteolytically processed by the MHC-bearing cell, the resulting antigenic peptide forms a complex with the antigen binding groove of the MHC molecule through various noncovalent associations. The MHC-peptide complex on the cell surface is recognized by a specific T cell receptor on a cytotoxic or helper T cell.

The highly polymorphic nature of these molecules arises in part from the multiple genetic loci encoding the molecules. The MHC of humans (also referred to as human leukocyte antigens (HLA)) on chromosome 6 has three loci, HLA-A, HLA-B and HLA-C, the first two of which have a large number of alleles encoding alloantigens. An adjacent region, known as HLA-D, is subdivided into HLA-DR, HLA-DQ and HLA-DP. The HLA region is now known as the human MHC region, and is equivalent to the H-2 region in mice. HLA-A, -B and -C resemble mouse H-2K, -D, and -L and are the class I MHC molecules. HLA-DP, -DQ and -DR resemble mouse I-A and I-E and are the class II molecules. MHC glycoproteins of both classes have been isolated and characterized (see, Fundamental Immunology (W. E. Paul, ed., 2nd Ed., 1989); and Roitt et al., Immunology (2nd Ed., 1989), which are both incorporated herein by reference).

Human class II MHC is a heterodimeric integral membrane protein. Each dimer consists of one α and one β chain in noncovalent association. The two chains are similar to each other, with the α chain having a molecular weight of 32-34 kD and the β chain having a molecular weight of 29-32 kD. Both polypeptide chains contain N-linked oligosaccharide groups and have extracellular amino termini and intracellular carboxy termini. The extracellular portions of the α and β chain that comprise the class II molecule have been subdivided into two domains of about 90 amino acids each, called α1, α2, and β1, β2, respectively. The α2 and β2 domains each contain a disulfide-linked loop. The α and β chains of class II molecules are encoded by different MHC genes and are polymorphic (see, Addas et al., Cellular and Molecular Immunology (2d Ed., 1994), which is incorporated by reference in its entirety).

The peptide-binding region of the class II molecule is formed by the interaction of the α1 and β1 domains. This interaction results in an open-ended, antigenic peptide-binding groove made up of two α helices, and an eight-stranded β-pleated sheet platform.

D. Identification of Peptides which Bind with High Affinity to Class II MHC Molecules

In one aspect, this invention comprises methods to identify polypeptides which are capable of competitively inhibiting antigens from binding to class II MHC molecules. Naturally occurring molecules which bind with high affinity to class II MHC molecules can be identified by Edman degradation (Falk et al., Nature, 351:290-294 (1991)), sequence analysis of naturally processed peptides (Rudensky et al., Nature, 353:622-626 (1991)), and tandem mass spectroscopy (Hunt et al., Science, 255:1261-1268 (1992)). Alternatively, methods used to screen for interacting proteins such as phage display, λ phage expression screening, and yeast two-hybrid screening can be adapted to identify high binding affinity peptide sequences. Finally, artificial sequences with known binding motifs can be synthesized or computational methods can be used to identify naturally occurring peptide sequences with known binding motifs and in vitro binding assays used to verify binding affinity.

In the preferred embodiment of this invention, peptides which bind with high affinity to HLA-DR2 class II MHC molecules are identified by phage display, a method which has recently emerged as a tool for defining the peptide binding characteristic of class II MHC molecules (Hammer et al., Ex. Med. 1007-1013 (1992)). Briefly, synthetic oligonucleotides are fused to M13 phage, where they are expressed as peptide:capsid fusion proteins. This random peptide library is incubated with class II MHC molecules. When the MHC molecules are captured, the bound phage which express interacting peptides are also pulled down. The recovered phage is amplified and selected again for binding to the class II MHC molecule. Three to four rounds of selection lead to the isolation and enrichment of phage which binds avidly to the class II MHC molecule. The identity of the peptide is determined by nucleotide sequencing.

E. Synthesis of Polypeptides

Peptides of this invention comprise those identified, e.g., by phage display, as binding particularly well to class II MHC HLA-DR2 molecules. Particularly illustrative polypeptides of the present invention comprise those set forth in SEQ ID NO:1, peptides that have post-translational modifications, or peptides with 90% identity to the polypeptide of SEQ ID NO:1.

Polypeptides of the invention can prepared using any of a variety of well known synthetic and/or recombinant techniques, which are both further described below.

1. Chemical Synthesis of Polypeptides

Polypeptides, portions and other variants generally less than about 150 amino acids can be generated by synthetic means, using techniques well known to those of ordinary skill in the art (see, Caruthers et al., Nucl. Acids Res. Symp. Ser. 215-223 (1980), Horn et al., Nucl. Acids Res. Symp. Ser. 225-232 (1980)). In one illustrative example, such polypeptides are synthesized using any of the commercially available solid-phase techniques, such as the Merrifield solid-phase synthesis method, where amino acids are sequentially added to a growing amino acid chain (see, Merrifield, J. Am. Chem. Soc. 85:2149-2146 (1963)). Equipment for automated synthesis of polypeptides is commercially available from suppliers such as Perkin Elmer/Applied BioSystems Division (Foster City, Calif.), and may be operated according to the manufacturer's instructions.

2. Synthesis by Recombinant Methods

Alternatively, the polypeptide compositions described herein are produced by recombinant methods (see, e.g., Sambrook et al., Molecular Cloning: A Laboratory Manual (2nd Ed., 1989); Maniatis et al., Molecular Cloning: A Laboratory Manual (1982); Current Protocols in Molecular Biology (Ausubel et al., eds., 1995)). Accordingly, this invention also comprises polynucleotide compositions that encode a polypeptide comprising the amino acid sequence of SEQ ID NO:1.

The present invention provides polynucleotide variants having substantial identity to the sequences disclosed herein in SEQ ID NO:1, for example 90%, 95%, 96%, 97%, 98%, or 99% or higher sequence identity compared to a polynucleotide sequence of this invention using the methods described herein, (e.g., BLAST analysis using standard parameters). One skilled in this art will recognize that these values can be appropriately adjusted to determine corresponding identity of proteins encoded by two nucleotide sequences by taking into account codon degeneracy, amino acid similarity, reading frame positioning and the like.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man. Furthermore, the polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

As will be understood by those of skill in the art, other recombinant constructions may be used to join sequences encoding a polypeptide of interest to nucleotide sequence encoding a fusion partner which enhances expression levels, increases solubility, and/or facilitates ease of synthesis, purification (e.g., affinity tags), or identification. A discussion of vectors which contain fusion proteins is provided in Kroll et al., DNA Cell Biol. 12:441-453 (1993).

One illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An polypeptide of the invention, when fused with this targeting signal, will associate more efficiently with class II MHC molecules and thereby efficiently prevent binding of antigens.

Fusion polypeptides may generally be prepared using standard techniques, including chemical conjugation. Natural, modified, or recombinant nucleic acid sequences may be ligated to a heterologous sequence to encode a fusion protein. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides. A peptide linker sequence may be employed to separate the first and second polypeptide components by a distance sufficient to ensure that each polypeptide folds into its secondary and tertiary structure.

One of skill would recognize that modifications can be made to the recombinant nucleic acids and fusion polypeptides without diminishing their biological activity. Some modifications may be made to facilitate the cloning, expression, or incorporation of the tag molecule into a fusion polypeptide. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Expression in Prokaryotes and Eukaryotes

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook et al., Molecular Cloning, A Laboratory Manual (1989), and Ausubel et al., Current Protocols in Molecular Biology (1989).

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the polynucleotide in host cells. A typical expression cassette thus contains a promoter operably linked to the nucleic acid sequence encoding the polypeptide and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322-based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a polynucleotide encoding the polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical—any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of the polypeptide. Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the polypeptide.

After the expression vector is introduced into the cells, the transfected cells are cultured under conditions favoring expression of the polypeptide, which is recovered from the culture using standard techniques identified below.

F. Protein Purification and Preparation

Encoded protein may be isolated and purified by standard methods including chromatography (e.g., high performance liquid chromatography, ion exchange, affinity, and sizing column chromatography), centrifugation, differential solubility, or by any other standard technique for the purification of proteins (see, generally, Scopes, Protein Purification (1982), Deutscher, Methods in Enzymology Vol. 182: Guide to Protein Purification. (1990)). The actual conditions used will depend, in part, on factors such as net charge, hydrophobicity, hydrophilicity, etc., and will be apparent to those having skill in the art. The functional properties may be evaluated using any suitable assays.

A number of procedures can be employed when recombinant polypeptides are being purified. For example, proteins having established molecular adhesion properties can be reversibly fused to the polypeptide. With the appropriate ligand, the polypeptide can be selectively adsorbed to a purification column and then freed from the column in a relatively pure form. The fused protein is then removed by enzymatic activity. Finally the polypeptide could be purified using immunoaffinity columns.

1. Purification of the Polypeptide from Recombinant Bacteria

Recombinant proteins can be expressed by transformed bacteria in large amounts, typically after promoter induction; but expression can also be constitutive. Promoter induction with IPTG is one example of an inducible promoter system. Bacteria are grown according to standard procedures in the art. Fresh or frozen bacteria cells are used for isolation of protein. Methods for isolating proteins from inclusion bodies and the periplasm are known to those of skill in the art.

2. Standard Protein Separation Techniques for Purifying Polypeptides

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the polypeptide can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The polypeptides can also be separated from other proteins on the basis of size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

The purified proteins may be further processed before use. For example, the proteins may digested with a specific enzyme to separate the fusion protein portions from the heterologous polypeptide.

3. Quantification of Proteins

Chemically synthesized and recombinant polypeptides can be measured based on the physical or functional properties of the product, including radioactive labeling of the product followed by analysis by gel electrophoresis, radioimmunoassay, ELISA, bioassays, etc.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton et al. Serological Methods, a Laboratory Manual (1990) and Maddox et al. J. Exp. Med. 158:1211-1216 (1983).

A wide variety of labels and conjugation techniques are known by those skilled in the art and may be used in various identification assays. Suitable reporter molecules or labels, which may be used include radionuclides, enzymes, fluorescent, chemiluminescent, or chromogenic agents as well as substrates, cofactors, inhibitors, magnetic particles, and the like.

G. Assays for Peptide Activity

The activity of the above-described polypeptides, polypeptide variants, and expression products of polynucleotides can be tested according to the assays described herein.

Once screening methods have been used to identify peptides which bind with high affinity to class II MHC molecules, a variety of in vitro binding assays known to those of skill in the art can be used to establish that the chemically or recombinantly synthesized versions of these peptides behave in a similar manner. In order to provide an optimal therapeutic effect, it is preferred that the peptide bind with an affinity on the order of other antigens, in particular, those implicated in autoimmune disease. In a preferred method, the ability of peptides to bind class II MHC is estimated by a competitive peptide binding assay. In an exemplary assay, purified HLA-DR2 is incubated with an excess of biotinylated antigen (e.g., myelin basic protein) in the presence or absence of test peptides. HLA-DR2 molecules are captured by adding the mixture to wells coated with anti-DR antibody or other means for HLA-DR2-specific binding. An alkaline phosphatase-linked streptavidin system is used to determine the amount of bound biotinylated antigen. In alternative embodiments, antigen molecules comprise other labels detectable by the addition of developing reagents or directly detectable label. Binding of the test peptide may also be directly assayed through the use of labeled peptides, rather than labeled antigen. The multi-component labeling systems and directly detectable labels are known in the art, and include fluorescent, colorimetric and radiolabels, for instance. One of skill in the art will recognize that the above-described assay can also be used to test in vitro binding of peptide to other HLA-DR alleles.

Assays measuring various aspects of the T cell response known to those of skill in the art can be used as a functional measure of the ability of test peptide to displace antigen and as an indicator of potential therapeutic efficacy for autoimmune disorders.

Briefly, an antigen of choice, either in the presence of absence of test peptide, can be mixed with responder cells, preferably established T cell lines or clones. Alternatively, responder cells may be peripheral blood mononuclear cells (PBMN) (a heterogeneous population including B and T lymphocytes, monocytes and dendritic cells), PBMNC lymphocytes, freshly isolated T lymphocytes, in vivo primed splenocytes, or cultured T cell. These cells can be obtained from mammals immunized with, or having a demonstrable cellular immune response to, the antigenic peptide and are selectively amplified and/or stimulated, thereby producing a subset of T cells that are specific for the antigenic peptide. For instance, antigenic peptide-reactive responder cells may be selected by flow cytometry, and particularly by fluorescence-activated cell sorting. This subset of responder cells can be maintained by repetitive stimulation with APCs presenting the same antigenic peptide. Alternatively, responder cell clones or lines can be established from this responder cell subset.

Activation of responder cells in response to the antigen of interest +/−peptide can be measured by several methods known to those of skill in the art. In a preferred embodiment, responder cell activation can be measured by the production of cytokines, such as IL-2. Cytokine production can be assayed by testing the ability of the antigen+responder cell culture supernatant to stimulate growth of cytokine-dependent cells. Alternatively, responder cell activation is determined by measuring proliferation using ³H-thymidine uptake (Crowley et al., J. Immunol. Meth. 133:55-66 (1990)) or by determining the presence of responder cell-specific, and particularly T cell-specific, activation markers using antibodies specific for such markers.

In an exemplary assay, the antigen of interest (e.g., MBP) is incubated with or without test peptide and transformed T cell clones in microtiter plates. Culture fluid is collected and assayed for γ-interferon and TNF-β cytokines by ELISA. The detection of γ-IFN by Ab ELISA has been described recently (Arimilli et al, J. Immunol. Methods 212:49-59 (1998)). The detection of TNF-β by ELISA is described in detail in the Example. Briefly, an anti-TNF-β antibody is incubated with goat anti-human TNF-β and this complex is detected by HRP-conjugated mouse anti-goat Ab and the substrate TMB. It will be obvious to those of skill of the art that other cytokines or proteins secreted in response to the T cell interaction with antigen can be assayed by ELISA using the numerous other detection systems. In another preferred method, T cell response is assayed by using a microphysiometer to measure production of acidic metabolites. See the Example for a detailed description of the protocol.

H. Animal Models

Similar assays and methods can be developed for and used in animal models. For instance, the therapeutic effect of a pharmaceutical composition of the polypeptide SEQ ID NO:1 or a polynucleotide encoding SEQ ID NO:1 can be tested in vivo in a number of animal models of HLA-DR-associated autoimmune disease. These diseases include, but are not limited to, insulin-dependent diabetes mellitus, multiple sclerosis, myasthenia gravis, pernicious anemia, rheumatoid arthritis, and systemic lupus erythematosus.

For example, NOD mice are a spontaneous model of IDDM. Treatment with the pharmaceutical compositions prior to or after onset of disease can be monitored by assay of urine glucose levels in the NOD mouse, as well as by in vitro T cell proliferation assays to assess reactivity to known autoantigens (see, e.g., Kaufman et al., Nature 366:69-72 (1993)) for example). Alternatively, induced models of autoimmune disease, such as EAE, can be treated with pharmaceutical composition. Treatment in a preventive or intervention mode can be followed by monitoring the clinical symptoms of EAE.

Following is a description of several other animal models of HLA-DR-associated autoimmune disease which can be used to assay in vivo effects of the peptide. It will be obvious to one of skill in the art that other suitable animal models for autoimmune diseases can be utilized in a similar manner.

Systemic Lupus Erythematosus (SLE)

F₁ hybrids of autoimmune New Zealand black (NZB) mice and the phenotypically normal New Zealand White (NZW) mouse strain develop severe systemic autoimmune disease, more fulminant than that found in the parental NZB strain. These mice manifest several immune abnormalities, including antibodies to nuclear antigens and subsequent development of a fatal, immune complex-mediated glomerulonephritis with female predominance, remarkably similar to SLE in humans (Knight et al., J. Exp. Med. 147:1653 (1978)), which is incorporated hereby by reference.

In both the human and murine forms of the disease, a strong association with MHC gene products has been reported. HLA-DR2 and HLA-DR3 individuals are at a higher risk than the general population to develop SLE (Reinertsen et al., N. Engl. J. Med. 299:515 (1970)), while in NZB/W F₁ mice (H-2^{d/u}), a gene linked to the h-2^{u} haplotype derived from the NZW parent contributes to the development of the lupus-like nephritis.

The effect of the invention can be measured by survival rates and by the progress of development of the symptoms, such as protenuria and appearance of anti-DNA antibodies.

Proteinuria can be measured by any method known to those of skill in the art, e.g. colorimetrically by the use of Uristix (Miles Laboratories, Inc., Elkhart, Ind.), giving an approximation of proteinuria as follows: trace, 10 mg/dl; 1+, 30 mg/dl; 100 mg/dl; 3+, 300 mg/dl; and 4+, 1000 mg/dl.

The presence of anti-DNA specific antibodies in NZB/W F₁ mice can be determined by using a modification of a linked immunosorbent assay (ELISA) described by Zouali et al., J. Immunol. Methods 90:105 (1986)) which is incorporated herein by reference.

Myasthenia Gravis (MG)

Myasthenia gravis is one of several human autoimmune diseases linked to HLA-D (Safenberg, et al., Tissue Antigens 12:136 (1978); McDevitt et al., Arth. Rheum. 20:59 (1977)) which are incorporated herein by reference. In MG antibodies to the acetyl choline receptors (AcChoR) impair neuromuscular transmission by mediating loss of AcChoR in the postsynaptic membrane.

SJL/J female mice are a model system for human MG. In these animals, experimental autoimmune myasthenia gravis (EAMG) can be induced by immunizing the mice with soluble AcChoR protein from another species. Susceptibility to EAMG is linked in part to the MHC and has been mapped to the region within H-2 (Christadoss et al., J. Immunol. 123:2540 (1979)).

AcChoR protein can purified from Toroedo californica and assayed according to the method of Waldor et al., Proc. Natl. Acad. Sci. USA 80:2713 (1983), incorporated by reference. For example, emulsified AcChoR, 15 μg in complete Freund adjuvant, is injected intradermally among six sites on the back, the hind foot pads, and the base of the tail. Animals are reimmunized with this same regimen 4 weeks later.

Evaluation can be made by measurement of anti-AcChoR antibodies by any method known to those of skill in the art, e.g., a microtiter ELISA assay as described in Waldor et al., supra. In an exemplary assay, the standard reagent volume is 50 μl per well. Reagents are usually incubated in the wells for 2 hr at RT. Five μg of AcChoR diluted in bicarbonate buffer, pH 9.6, is added to each well. After incubation with AcChoR, the plates are rinsed four times with a wash solution consisting of phosphate-buffer saline containing 0.05% Tween and 0.05% NaN₃. Mouse sera are diluted in 0.01M PBS (pH 7.2), 1.5 mfr MgCl₂, 2.0 mM 2-mercaptoethanol, 0.05% Tween-80, 0.05% NaN₃ (p-Tween buffer) and incubated on the plate. After the plate is washed, beta-galactosidase-conjugated sheep anti-mouse antibody diluted in P-Tween buffer is added to each well. After a final washing, the enzyme substrate, p-nitrophenylgalctopyranoside is added to the plate, and the degree of substrate catalysis is determined from the absorbance at 405 nm after 1 hr.

Anti-AcChoR antibodies are expected to be present in the mice immunized with AcChoR as compared to nonimmunized mice. Treatment with complex is expected to significantly reduce the titer of anti-AcChoR antibodies in the immunized mice.

The effect of treatment with the invention on clinical EAMG can also be assessed by any method known to those of skill in the art. Myasthenia symptoms include a characteristic hunched posture with drooping of the head and neck, exaggerated arching of the back, splayed limbs, abnormal walking, and difficulty in righting. Mild symptoms are present after a standard stress test, and should be ameliorated by administration of complex.

Rheumatoid Arthritis (RA)

In humans, susceptibility to rheumatoid arthritis is associated with HLA D/DR. The immune response in mice to native type II collagen has been used to establish an experimental model for arthritis with a number of histological and pathological features resembling human RA. Susceptibility to collagen-induced arthritis (CIA) in mice has been mapped to the H-2 I region, particularly the I-A subregion (Huse et al., Fed. Proc. 43:1820 (1984)).

Mice from a susceptible strain, DEA-1 can be caused to have CIA by treatment of the mice with native type II collagen, using the technique described in Wooley et al., J. Immunol. 134:2366 (1985), incorporated herein by reference.

In another model adjuvant arthritis in rats is an experimental model for human arthritis, and a prototype of autoimmune arthritis triggered by bacterial antigens (Holoschitz et al., Prospects of Immunology (1986); Pearson, Arthritis Rheum. 7:80 (1964)). The disease is the result of a cell-mediated immune response, as evidenced by its transmissibility by a clone of T cells which were reactive against the adjuvant (MT); the target self-antigen in the disease, based upon studies with the same cloned cells, appears to be part(s) of a proteoglycan molecule of cartilage.

Adjuvant disease in rats is produced as described by Pearson supra, i.e., by a single injection of Freund's adjuvant (killed tubercle bacilli or chemical fractions of it, mineral oil, and an emulsifying agent) given into several depot sites, preferably intracutaneously or into a paw or the base of the tail. The adjuvant is given in the absence of other antigens.

The effect of the invention treatment on manifestations of the disease can be monitored by any method known to those of skill in the art. These manifestations are histopathological, and include an acute and subacute synovitis with proliferation of synovial lining cells, predominantly a mononuclear infiltration of the articular and particular tissues, the invasion of bone and articular cartilage by connective tissue pannus, and periosteal new bone formation, especially adjacent to affected joints. In severe or chronic cases, destructive changes occur, as do fibrous or bony ankylosis. These histopathological symptoms are expected to appear in control animals at about 12 days after sensitization to the Freund's adjuvant.

Insulin Dependent Diabetes Mellitus (IDDM)

IDDM is observed as a consequence of the selective destruction of insulin-secreting cells within the Islets of Langerhans of the pancreas. Involvement of the immune system in this disease is suggested by morphologic evidence of early infiltration of the Islets by mononuclear cells, by the detection of anti-islet cell antibodies, by the high frequency of HLA-DR3 and -DR4 alleles in IDDM populations, and by clinical associations between IDDM and various autoimmune diseases. An animal model for spontaneous IDDM and thyroiditis has been developed in the BB rat. As in humans, the rat disease is controlled in part by the genes encoding the MHC antigens, is characterized by islet infiltration, and is associated with the presence of anti-islet antibodies. The I-E equivalent class II MHC antigens appear to be involved in manifestation of the autoimmune diseases in the BB rat. Biotard et al., Proc. Natl. Acad. Sci. USA 82:6627 (1985).

In morphologic evaluation, insulitis is characterized by the presence of mononuclear inflammatory cells within the islets. Thyroiditis is characterized by focal interstitial lymphocytic infiltrate within the thyroid gland, as a minimum criterion. Most severe cases show diffuse extensive lymphocytic infiltrates, disruption of acini, fibrosis, and focal Hurthle call change. See Biotard et al. supra.

Treatment of the BB rats with the invention is expected to ameliorate or prevent the manifestation of the clinical and morphological symptoms associated with IDDM and thyroiditis.

In another model, the NOD mouse strain (H-2K^{d} D^{b}) is a murine model for autoimmune IDDM. The disease in these animals is characterized by anti-islet cell antibodies, severe insulitis, and evidence for autoimmune destruction of the beta-cells (Kanazawa, et al., Diabetolooia 27:113 (1984)). The disease can be passively transferred with lymphocytes and prevented by treatment with cyclosporin-A (Ikehara et al., Proc. Natl. Acad. Sci. USA 82:7743 (1985)); Mori et al., Diabetolooia 29:244 (1986). Untreated animals develop profound glucose intolerance and ketosis and succumb within weeks of the onset of the disease. Seventy to ninety percent of female and 20-30% of male animals develop diabetes within the first six months of life. Breeding studies have defined at least two genetic loci responsible for disease susceptibility, one of which maps to the MHC. Characterization of NOD Class II antigens at both the serologic and molecular level suggest that the susceptibility to autoimmune disease is linked to I-AB (Acha-Orbea and McDevitt, Proc. Natl. Acad. Sci. USA 84:235 (1907)).

Treatment of Female NOD mice with complex is expected to lengthen the time before the onset of diabetes and/or to ameliorate or prevent the disease.

Experimental Allergic Encephalomyelitis (EAE)

Experimental allergic encephalomyelitis (EAE) is an induced autoimmune disease of the central nervous system which is a model for multiple sclerosis (MS). The disease can be induced in many species, including mice and rats.

The disease is characterized by the acute onset of paralysis. Perivascular infiltration by mononuclear cells in the CNS is observed in both mice and rats. Methods of inducing the disease, as well as symptomology, are reviewed in Aranson, The Autoimmune Diseases (Rose and Mackay, eds., 1985), and in Acha-Orbea et al., Ann. Rev. Imm. 7:377-405 (1989).

One of the genes mediating susceptibility is localized in the MHC class II region (Moore et al., J. Immunol. 124:1815-1820 (1980)). The best analyzed encephalitogenic protein is myelin basic protein (MBP), but other encephalitogenic antigens are found in the brain. The immunogenic epitopes have been mapped (see, Acha-Orbea et al., supra.). In the PL mouse strains (H-2^{u}) two encephalitogenic peptides in MBP have been characterized: MBP peptide p35-47 (MBP 35-47), and acetylated NSF p1-9 (MBP 1-9).

The effect of the invention on ameliorating disease symptoms in individuals in which EAE has been induced can be measured by survival rates, and by the progress of the development of symptoms.

I. Pharmaceutical Compositions and Administration

Pharmaceutically acceptable carriers are determined in part by the particular composition being administered (e.g., nucleic acid, protein, transfected cell) as by the particular method used to administer the composition. Accordingly, there are a wide variety of suitable formulations of pharmaceutical compositions of the present invention (see, e.g., Remington's Pharmaceutical Sciences (17^{th} ed., 1989). Administration can be in any convenient manner, e.g., by injection, oral administration, inhalation, or transdermal application.

Formulations suitable for oral administration can consist of (a) liquid solutions, such as an effective amount of the packaged nucleic acid or polypeptide suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into the subjects. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the compositions disclosed herein. The formation and use of liposomes is generally known to those of skill in the art (see, for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516, specifically incorporated herein by reference in its entirety). Further, various methods of liposome and liposome-like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety).

Liposomes have been used effectively to introduce genes, drugs (Heath & Martin, 1986; Heath et al., 1986; Balazsovits et al., 1989; Fresta & Puglisi, 1996), radiotherapeutic agents (Pikul et al., 1987), enzymes (Imaizumi et al., 1990a; Imaizumi et al., 1990b), viruses (Faller & Baltimore, 1984), transcription factors and allosteric effectors (Nicolau & Gersonde, 1979) into a variety of cultured cell lines and animals. In addition, several successful clinical trials examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori & Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the compositions. They are widely suitable as both water- and lipid-soluble substances can be entrapped, i.e., in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977, 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution. However, a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for hours or days, depending on their composition, and half lives in the blood range from minutes to several hours. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominant site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety).

J. Methods to Treat Autoimmune Disease

In further aspects of the present invention, the pharmaceutical compositions described herein may be used for the treatment of HLA-DR-associated autoimmune disease. Within such methods, the pharmaceutical compositions described herein are administered to a patient, typically a warm-blooded animal, preferably a human. A patient may or may not be afflicted with an autoimmune disease. Accordingly, the above pharmaceutical compositions may be used to prevent the development of autoimmune disease or to treat a patient afflicted with an autoimmune disease.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. In general, an appropriate dosage and treatment regimen provides the active compound(s) in an amount sufficient to provide therapeutic and/or prophylactic benefit. An “effective amount” of such a pharmaceutical composition is an amount that provides a clinically significant decrease in a deleterious T cell-mediated immune response to an autoantigen, for example, those associated with IDDM, or provides other pharmacologically beneficial effects. Such a response can be monitored by establishing an improved clinical outcome in treated patients as compared to non-treated patients. “Effective amounts” will depend, in part, on the particular condition to be treated, age, weight, and general health of the patient, and other factors evident to those skilled in the art. Preferably the amount of the peptide administered will be within the range of 20-80 μg/kg. Compounds having significantly enhanced half-lives may be administered at lower doses or less frequently.

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Vaccines should be capable of causing an immune response that leads to an improved clinical outcome in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

Kits can also be supplied for therapeutic uses. Thus, the subject composition of the present invention may be provided, usually in a lyophilized form, in a container. The peptide is included in the kits with instructions for use, and optionally with buffers, stabilizers, biocides, and inert proteins. Generally, these optional materials will be present at less than about 5% by weight, based on the amount of peptide, and will usually be present in a total amount of at least about 0.001% by weight, based on the peptide. It may be desirable to include an inert extender or excipient to dilute the active ingredients, where the excipient may be present in from about 1 to 99% weight of the total composition.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

The following example is provided by way of illustration only and not by way of limitation. Those of skill in the art will readily recognize a variety of noncritical parameters that could be changed or modified to yield essentially similar results.

### EXAMPLE

### Identification of a Motif for the HLA-DR2 Binding Peptides Using a Phage Display Library

A. Introduction

Class II MHC molecules are heterodimeric cell surface proteins that bind peptide fragments of proteins and display these peptides for recognition by CD4+ T cells (Schwartz, Annu. Rev. Immunol, 237-252 (1985)). Different alleles of class II MHC molecules bind unique families of peptides. Even within each family of class II MHC molecules a large number of diverse peptides are bound in order to guarantee T cell immunity to a wide variety of antigens.

Different approaches have been used in order to understand and define the structural requirements for the interaction between the peptides and class II MHC molecules. They include the Edman degradation method (Falk et al., Nature 351:290-294 (1991)) sequence analysis of naturally processed peptides (Rudensky et al., Nature 353:622-626 (1991)), and tandem mass spectroscopy (Hunt et al., Science 255:1261-1268 (1992)).

Recently phage display has emerged as yet another tool to define the peptide binding characteristics to MHC Class II molecules (Hammer et al., Ex. Med. 1007-1013 (1992)). In our approach we used an M13 peptide library composed of 10×10⁶ 12-mers to screen for phages that bind to HLA-DR2*0101 allele. Our goal was to define the motifs used for binding to the *0101 allele. One peptide derived from this library bound to the class II MHC molecule with an affinity equal to that of one of the known strong binding peptides. The peptides isolated show some similarity to other class II MHC binding peptides but with enough differences to suggest a diversity in peptide binding motifs for different class II MHC molecules.

B. Materials and Methods

Reagents and Strains

M13 mp19 and XL1Blue strain were obtained from Stratagene, Inc. (La Jolla, Calif.). The phage display plasmid was from New England Biolabs. Peptides were synthesized with a Miligen peptide synthesizer using Fmoc chemistry and were purified by Reverse Phase HPLC. Homogeneity of each peptide was confirmed by analytical RP-HPLC.

Purification and Biotinylation of HLA-DR2

DR2 molecules were purified from the human EBV-B cell line. E of Mab L243 (IgG2a) affinity chromatography. A 10 μM solution of affinity purified DR2 was biotinylated with 200 μM Biotin-XX-NHS in 0.25 NaHCO3/0.2% NP-40 for 1 hr. at room temperature.

Construction of the M13 Peptide Library

The M13 plasmid with a degenerate 12-mer oligonucleotide was bought from New England Biolabs and the DNA was transformed by electroporation into E. coli SCSI cells. Ten transformations each containing 50 ml of cells and 200 ng of DNA were performed to produce 20 million independent clones. The library was amplified in LB/ampicillin media as described. The overall amplification factor was 10⁴.

M13-DR2 Binding Assay

The binding assays were carried out as previously described (Nag et al., J. Bio. Chem. 10413-10418 (1996)). Briefly, a 100 million phages displaying peptides of random sequence were mixed with a similar number of M13 mp19 as reference and incubated with about 20 pmol biotinylated DR2*0101 in binding buffer (50 mM Tris-HCl, pH 7.5, 150 mM NaCl, 1 mM EDTA, 1 mM PMSF and 0.2% NP-40). After a 24 hr. incubation, ESA blocked streptavidin on 4% beaded agarose was added and incubated for 10 mins. The phage-DR2 complex was purified by washing the solid phase several times with binding buffer. The phages were eluted with elution buffer (0.1N glycine-HCl, pH 2.2, 1 mg/ml BSA) for 10 minutes and neutralized with 2 M Tris base. The ratio of the phages displaying peptides to M13 mp19 phages was determined in the initial mixture and the eluates by plating both on X-Gal indicator plates. An enrichment of white (phage-displaying peptides) versus blue plaques (M13 mp19) indicated peptide-based binding.

Library Screening

Up to a billion phages of the amplified M13 peptide library were incubated with DR2 under the same conditions as described for the M13/DR2 binding assay. The phage eluates were concentrated and washed with 150 mM NaCl/50 mM Tris-HCI, pH 7.5 by ultradialysation (Microsep Microconcentrator, 30K cutoff, Filtron Technology Corp., Northbourough, Mass.). Up to 10 million phages were eluted and used to infect 500 μL of E. coli XL1blue plating cells (OD=1). The infected cells were transferred to 7 ml LB media in a 25 cm² tissue culture flask. After 1 hr. incubation at 37° C., 40 μg/ml ampicillin was added. After overnight incubation, the amplified phages were harvested and purified twice with polyethylene glycol. The screening procedure was repeated three times and the eluate from the fourth round was used to isolate and sequence individual clones.

Synthesis of Various MBP Peptides

All peptides prepared for these studies were acetylated at the N-terminus and amidated at the C-terminus. The MBP (83-102) Y83 peptide with the sequence Ac-YDENPVVHEKMVTPRTPP and the MBP (124-143) peptide with the sequence Ac-GFGYGGRASDYKSAHKGFKG were synthesized by the standard solid phase method using side-chain protected Fmoc amino acids on an Applied Biosystems 431A automated peptide synthesizer. The deprotected, crude peptides were purified by reverse-phase HPLC, and the homogeneity and identity of the purified peptides were confirmed by mass spectrometry. All terminally truncated and alanine analog MBP peptides were synthesized by solid phase peptide synthesis using an ABIMED/GILSON multiple peptide synthesizer by Fmoc chemistry. All chemicals, including the Rink amide MBHA resin and side chain protected Fmoc amino acids were obtained from Nova Biochem, San Diego, Calif.

The Peptide/DR2 Binding Assay

The ability of peptides to bind HLA-DR2 was estimated by competitive peptide binding assay. Purified HLA-DR2 (2 μg/ml) was incubated with 50-fold excess Bt-MBP 83-102Y83 peptide in the presence or absence of test peptides. The ratio of Et-MEP 831-2Y83 peptide to the test peptide was 1:1. After incubation at 37° C. for 48 hrs, the complex was added to the wells coated with anti-DR antibody and incubated for 2 hours at 37° C. The wells were then washed and alkaline phosphatase-linked streptavidin was added and the reaction incubated for 1 hour at 37° C. The excess streptavidin was washed and the ELISA color was developed by using p-nitrophenol phosphate in 0.1M diethanolamine as substrate. The test peptide was considered to be the stronger binding peptide if the O.D was less than the O.D of Bt 83-102Y83 peptide in the absence of the test peptide.

T Cell Receptor Occupancy Assay

The Herpes saimiri virus (HSV) transformed SS8T human T cell clone was a generous gift from Dr. H. Wekerle. The clone is restricted to DR2 (DRB5*0101) and MBP (84-102) peptide. It was cultured in RPMI 1640 medium supplemented with 2 mM L-glutamine, 100 units/ml penicillin, 100 μg/ml streptomycin, 10% fetal bovine serum and 50 units/ml human recombinant IL-2 (rIL-2) at 37° C. (culture medium). Every alternate day, cells were transferred to fresh culture medium. Various peptides at different concentrations were incubated with the cells in a microtiter tissue culture plate at a density of 20,000 cells/200 μl/well in the absence of rIL-2. After 48 h of incubation at 37° C., the culture fluids were collected from each well to test for γ-IFN and TNF-β cytokines. The detection of γ-IFN was by Ab ELISA as described recently (Arimilli et a.l, J. Immunol. Methods 212:49-59 (1998)). For the detection of TNF-β, Nunc Maxisorb 96-well plates were coated with anti-human TNF-β mAb at a concentration of 0.5 μg/well and incubated at 4° C. overnight. The wells were blocked with 0.1% bovine serum albumin, and samples were incubated at RT for 2 h. A standard curve was generated by using recombinant human TNF-β with a dilution range of 500 to 0.01 ng/ml. Goat anti-human TNF-β was then added at a concentration of 1 μg/ml and plates were incubated at 25° C. for an additional 2 h. Wells were washed three times and incubated with HRP-conjugated mouse anti-goat Ab at a concentration of 1 μg/ml for 1 h at 25° C., prior to developing color using TMB as a substrate. The reaction was stopped by 2N sulfuric acid at 5 mm, and the absorbance was measured at 450 nm.

Measurement of T Cell Metabolic Acidification Rate

Freshly cultured SS8T cells were immobilized into microphysiometer cell capsules as described earlier (Arimilli et al., J. Immunol. Methods 212:49-59 (1998)) using low-melt agarose (Molecular Devices Corp., Sunnyvale, Calif.). Briefly, cells were rested from IL-2 pulsing for 2 days. Cells were counted and suspended in a serum-free loading medium (low-buffering RPMI 1640 containing 10% fatty acid free, endotoxin-free BSA). Cells were collected by centrifugation and resuspended at a concentration of 3×10⁵ T cells per 7.5 μl of loading medium. Low-melt agarose (Molecular Devices Corp.), melted and stored at 37° C., was added to the suspended cells to a concentration of 2.5 μl per 7.5 μl. Ten μl of the agarose/cell mixture was immediately spotted into the center of the cell capsule cups (Molecular Devices Corp.) held in a 12 well culture plate. After 5 minutes, 2 ml of loading medium was placed in the capsule cup over the solidified agarose and a membrane insert was placed over the cells. The assembled cell capsule was loaded in the Cytosensor chamber at 37° C. and perfused at 50 μl per min with low-buffering RPMI 1640 medium containing 10% of BSA per ml but no added HEPES or bicarbonate. Extracellular acidification measurements were made in the Cytosensor microphysiometer by collecting potentiometric measurements for 45 seconds every 2 mm (Arimilli et al, Journal of Immunological Methods, 212, 49-59 (1998)). Acidification rate data (uV/sec) were normalized to 100% prior to cell stimulation, which allowed for comparison of data from cells in separate chambers.

C. Results

Isolation of Phages and Binding to the DR2*0101 Allele

A M13 peptide library with 20 million independent phages was generated. After four rounds of successive enrichment and selection, the phages were eluted. In order to monitor for enrichment for binding to DR2*0101, a colorometric assay was used with the wild type phage (blue) used a background versus the peptide-bearing phage (white). The lower the background of blue phages, the more specific the binding to the DR2 molecule. By the second round of screening there were no blue plaques visible. After the final round, the phages were isolated and sequenced. There were two distinct “families” of peptides (Table 1)

One corresponding peptide from each family was synthesized and binding assays were carried out. It was shown that one peptide from one of the families did bind DR2 within the same range of affinity as the MBP 84-102 peptide.

TABLE 1
Amino Acid sequences of the random peptides
generated by the DR2*0101 selected phage:

F H W S W Y P P R Q A S
L P T S F N P P D G F T
T Q N H M L N L A P W T
V V P K P A S Q M L N T
Q I P P E Q R S L F E I
G H I K S S I S F M P M
G H I K S S I S F M G M
A H L R S T L S Y L P L
S H L R S S L T W S S I
S H L R S T L S P D P R
A H L H S N I S C S G C
T H F P T M T S R L S I
A R I H T S T I E A A L

Sequence Motifs of the Peptides

After the final round of DR2 binding and washing, the phages were isolated and sequenced. The isolated twenty phages could be broken up into two groups. The first group of peptides was enriched for aromatic residues in the N terminus of the peptide but otherwise there was no distinct pattern. The N-terminus of the second group of peptides had very strong sequence homology. In most other class II MHC binding peptides, an aromatic residue occupies the P1 position of the peptide pocket of the Class II molecule. In this group of peptides, there were no aromatic residues near the N terminal end. There was a very strong homology for a histidine residue at this position, most likely because histidine shares many properties of the aromatic residues like spatial bulk and even some properties of aromaticity. Immediately following the histidine residue was a very hydrophobic residue, which was usually filled by a leucine or an isolecucine. There were then two serines following the lysine in most of the peptides. There was a leucine or an isolecucine residue at the seventh position, which satisfies a requirement for a hydrophobic amino acid at the P4 position (Sinagaglia et al., Curr. Opin. Immunol. 6:52-56 (1994)). Immediately following the P4 position was a serine followed by an aromatic residue. The tail end residues did not show any homology with one another.

Peptide Binding Assays

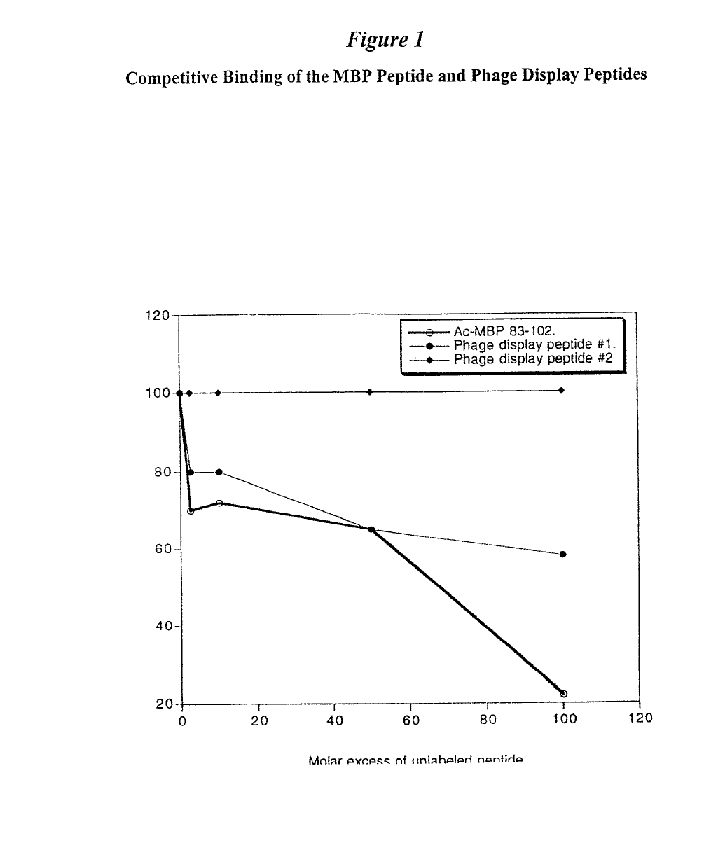

The specificity of the binding of two of the phage display peptides to DR2 was demonstrated in a competitive binding experiment. Purified DR2 was incubated with 50-fold molar excess of biotinylated excess MBP 83-102Y83 peptide in the presence of non-biotinylated MBP 83-102Y83 and the phage peptides. As shown in FIG. 1, increasing concentrations of peptide #1 inhibited the binding of biotinylated MBP 83-102 to the same extent as the unbiotinylated MBP 83-102 peptide. This suggests that the binding of the phage display peptide #1, GHIKSSISFMGM, is comparatively in the same range as that of the MBP 83-102 peptide. The phage display peptide #2 did not seem to bind competitively.

The T Cell Assay

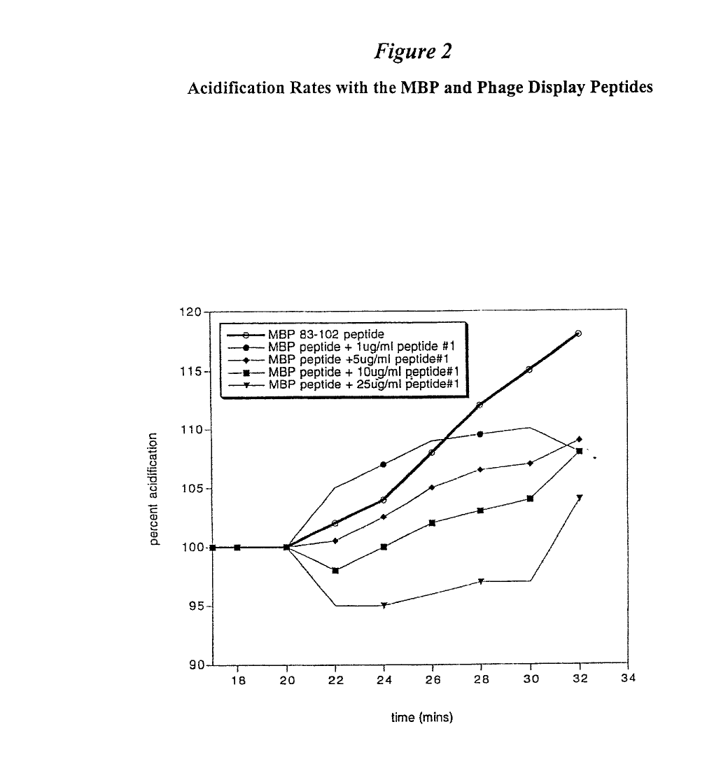

The recognition of the MBP peptides as well as the phage display peptide #1, GHIKSSISFMGM, was performed using herpes saimiri virus-transformed SS8T cloned human T cells. The SS8T cell clone was generated from an MS patient and was characterized for its restriction to HLA-DR2 and MBP 84-102 (Weber et al., PNAS USA 90 (1993)). The TCR engagement was monitored by an increase in γ-IFN cytokine in a dose-dependent manner. Such an increase in γ-IFN production by T cells was correlated with the occupancy of TCRs in an earlier study (Weber et al., PNAS USA 90 (1993)). Human T cells are known to express low levels of class II MHC molecules and can be stimulated in the presence of antigenic peptides. As shown in FIG. 2, a specific increase in γ-IFN production was noted when the SS8T cells were exposed to the MBP 83-102 peptide. In various controls, cells exposed to either the MBP 124-143 peptide or the MBP 1-14 peptides did not show any increase in γ-IFN production.

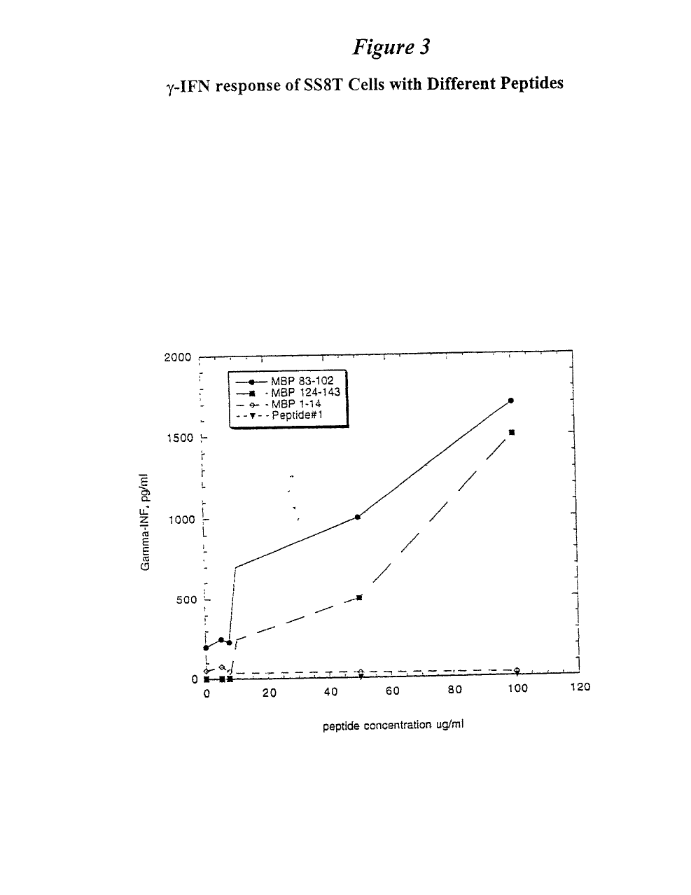

T cell stimulation assays were carried out to see if these peptides could stimulate MBP 83-102 specific T cell hybridomas. SS8T cells were immobilized in agarose and exposed to both the phage display peptides as well as the MBP 83-103,Y83 peptide. As FIG. 3 shows, the MBP 83-102Y83 peptide showed an increased level of extracellular acidification rate within 10 minutes of exposure to the SS8T cells. In contrast, the phage display peptides did not stimulate the SS8T cells but instead act like a competitive inhibitor. Increasing concentrations of the phage display peptide #1 brought about decreasing activation of the hybridomas in a competitive manner.

D. Discussion

Most of the information that we possess regarding class II MHC binding peptides is derived from isolating and then sequencing naturally binding peptides. These peptides originate from cellular proteins, primarily from the plasma membrane. Based on this information, several class II MHC peptide binding motifs have been proposed. However, many of these peptides are not very strong binders, with the kD of binding in the high nM range. Recently, phage display was used to generate a library of peptides to identify high affinity binding peptides (Hammer et al., Ex. Med. 1007-1013 (1992)). The advantage of this approach is that the peptides are randomly generated and the peptides can be chosen on the basis of the affinity of binding to the class II MHC molecule. Thus only the high affinity peptides are chosen and one can get a better idea of the binding motif for a class II MHC molecule. The drawback to this approach is that the peptides generated by the phage are 9-mers and therefore might be too small for the class II MHC molecules which possess a core binding region of 7-10 residues. It is well established that the flanking residues playing a key role in peptide affinity with the central 5-8 residues contributing the majority of the contacts.

For our experiments, we chose a phage with a library of 12-mer peptides. This peptide length was long enough to give us a better idea of the class II MHC binding motif yet small enough such that a large library of peptides could be generated.

Two DR2*0101 allele peptide binding motifs were identified after sequencing the 15 clones at the end of the screening. All the peptides showed a very strong sequence homology near the N-terminus of the peptide and very little near the C terminus, suggesting that the N terminus of the peptide plays a stronger role in the binding of the class II MHC molecule. Of the peptides sequenced, there appeared to be one group which shared a very strong sequence homology. Further peptide binding studies clarified that it was this group of peptides which reflected the binding affinities in vivo better. This group of peptides shared some similarities to the peptide binding motifs of known class II MHC molecules. The P4 position of the peptides showed a remarkable enrichment for serines, unlike the natural peptides isolated from *0101 allele in which there is a requirement for a hydrophobic residue. The P6 residue showed an enrichment for hydrophobic residues.

Apart from these anchor residues, there appears to be a number of other residues that play an important role in Class II binding. Following the P1 position is a residue which is occupied almost 90% of the time with a hydrophobic amino acid. Immediately afterwards is a positive charged amino acid. This position is occupied by a lysine or an arginine about 60% of the time. The presence of two serines following the charged residues is quite common in the peptides binding to the *040 1 alleles and has been hypothesized to play a role in distinguishing alleles (Sinagaglia et al., Curr. Opin. Immunol., 6:52-56 (1994)). However, they seem to predominate in peptide binding to the *0101 allele.

Our studies, like those of Hammer et al., Cell 74:197-203 (1993) also show that certain residues tend to be underrepresented in DR2 binders. In particular, the negative residues Glu and Asp were present very infrequently in the C terminus end of the peptides and absent from any of the binding regions. This confirms earlier studies, where it was concluded that in addition to anchor residues, the overall composition of a given peptide is important for MHC binding (Sinagaglia et al., Curr. Opin. Immunol. 6:52-56 (1994)).

Recent works have proposed certain class II MHC binding motifs (for review see Sinagaglia et al., Curr. Opin. Immunol. 6:52-56 (1994)). The proposed motifs call for an aromatic or hydrophobic residue at the amino terminus, a relatively small residue at position 6, and a relatively hydrophobic residue at position number 9. Our data are in substantial agreement with those results, except for the presence of the hydrophobic residue, isoleucine, at position 6. However, Hammer et al., Cell 74:197-203 (1993) have reported that this position may play a role in distinguishing alleles.

All publications and patent applications cited in this specification are herein incorporated by reference as if each individual publication or patent application were specifically and individually indicated to be incorporated by reference.

What is claimed is:

1. An isolated polypeptide comprising an amino acid sequence of SEQ ID NO:1.

2. The polypeptide of claim 1, wherein the polypeptide is from 50 to 100 amino acids in length.

3. The polypeptide of claim 1, wherein the polypeptide is from 25 to 50 amino acids in length.

4. The polypeptide of claim 1, wherein the polypeptide has an amino acid sequence of SEQ ID NO:1.

5. The polypeptide of claim 1, wherein the polypeptide comprises post-translational modifications.

6. A pharmaceutical composition comprising a pharmaceutically acceptable carrier and polypeptide comprising an amino acid sequence of SEQ ID NO:1.

7. A pharmaceutical composition according to claim 6, wherein said composition further comprises a member selected from the group consisting of:
a) a liposome,
b) a nanocapsule, and
c) a microparticle.

8. A method of treating autoimmune disease in a subject, the method comprising the step of administering to said subject a therapeutically effective amount of an isolated polypeptide comprising an amino acid sequence of SEQ ID NO:1.

9. A method of treating autoimmune disease according to claim 8, wherein said subject is a human.

10. A method of treating autoimmune disease according to claim 8, wherein said autoimmune disease is associated with HLA-DR class II MHC molecules.

11. A method of treating autoimmune disease according to claim 10, wherein said HLA-DR class II MHC molecules are HLA-DR2 molecules.

12. A method of treating autoimmune disease according to claim 8, wherein said autoimmune disease is a member selected from the group consisting of:
a) insulin-dependent diabetes mellitus,
b) multiple sclerosis,
c) myasthenia gravis,
d) pernicious anemia,
e) rheumatoid arthritis, and
f) systemic lupus erythematosus.

13. A method of treating autoimmune disease according to claim 8, wherein said polypeptide is administered with a pharmaceutically acceptable carrier.

14. A method of treating autoimmune disease according to claim 8, wherein said polypeptide comprises an epitope that competes with the myelin basic protein (MBP) protein for binding.

15. A method for treating autoimmune disease according to claim 8, wherein said polypeptide comprises an epitope that antagonizes the T cell response induced by myelin basic protein (MBP) protein.

16. An isolated polypeptide comprising an amino acid sequence having at least 90% identity to the sequence provided in SEQ ID NO:1.

17. A polynucleotide encoding a polypeptide comprising the amino acid sequence of SEQ ID NO:1.

18. A method of treating autoimmune disease in a subject, the method comprising the step of administering to the subject a therapeutically effective amount of a polynucleotide according to claim 17.
